(19) United States
(12) Patent Application Publication
(10) Pub. No.: US 2010/0035832 A1
(43) Pub. Date: Feb. 11, 2010

(54) Macrolides

(75) Inventors: Audun Heggelund, Oslo (NO); Kjell Undheim, Sandvika (NO)

Correspondence Address: CANTOR COLBURN, LLP, 20 Church Street, 22nd Floor, Hartford, CT, 06103, US

(73) Assignee: ALPHARMA APS, Copenhagen S. (DK)

(21) Appl. No.: 11/813,682

(22) PCT Filed: Jan. 12, 2006
(86) PCT No.: PCT/EP2006/000336

§ 371 Date: Jul. 11, 2007

(30) Foreign Application Priority Data
Jan. 12, 2005 (DK) PA2005 00060
Mar. 17, 2005 (DK) PA2005 00390

(51) Int. Cl. A61K 31/7048; C07H 17/08; A61P 31/00

(52) U.S. Cl. 514/28; 536/7.1

(57) ABSTRACT

This invention relates to a novel class of antibiotic agents, more specifically to macrolides comprising an oxazolidinone structure, their preparation, pharmaceutical compositions containing them, their use and methods of treatment using them.

### FIELD OF INVENTION

This invention relates to a novel class of antibiotic agents, more specifically to macrolides comprising an oxazolidinone structure, their preparation, pharmaceutical compositions containing them, their use and methods of treatment using them.

### BACKGROUND OF INVENTION

The development of bacterial resistance to currently available antibacterial agents is a growing global health problem. Hence extensive work is going on to discover novel classes of antibacterial agents possessing new mechanisms of action. Such agents would exhibit a lack of cross-resistance with existing antimicrobial drugs.

The macrolide binding site in bacteria has been shown to be located in the 50S ribosomal subunit inside the protein exit tunnel near the peptidyl transferase site. Therein the macrolides constitute a molecular block for the growing chain for protein production once the peptide chain has reached a certain length.¹

Other structurally unrelated antibiotics such as lincosamides and streptogramin B also exert their action at this site. Bacterial mutations in this common binding site lead to combined resistance towards all three antibiotics, so called MLS_{B}-resistance.²

Macrolide antibiotics are disclosed in several patents, such as U.S. Pat. No. 6,590,083 B1, EP 248279 A2, U.S. Pat. No. 5,635,485 A, WO 9809978 A, WO 9854197, etc. FR 2692579 A, EP 487411 A, EP 680967 A, EP 606024 A1, EP 596802 B1, U.S. Pat. No. 5,527,780 A, and U.S. Pat. No. 6,399,582 B1 relate to derivatives of erythromycin having an 11,12 oxazolidinone group substituted on the N-11 atom with e.g. aralkyl or heteroarylalkyl. The teaching of these patents is that there should be an aliphatic linker between N-11 and the phenyl group, said linker having 4-6 carbon atoms, cf. table I in U.S. Pat. No. 6,399,582 and example 39 in U.S. Pat. No. 5,527,780.

FR 2692579 A, EP 487411 A, EP 596802 A1, EP 606024 A1, EP 680967 A, and U.S. Pat. No. 5,527,780 A disclose macrolides having a oxazolidinone structure. However, none of these patent documents disclose macrolides having a substituted phenyl ring bound directly to the nitrogen atom of the oxazolidinone structure, and none disclose macrolides having the phenyl ring bound directly to a saturated ring containing at least one nitrogen atom.

### SUMMARY OF INVENTION

The objective underlying the present invention is to provide novel macrolides with antibacterial activity. This objective has been met by the macrolides of the invention as defined in the claims. Futhermore, it has surprisingly turned out that an aliphatic linker between N-11 and the parasubstituted phenyl ring is not needed.

In its broadest scope, the invention relates to 11-deoxy-11-aminomacrolides wherein the 11-amino and the 12-oxy groups are joined by a common carbonyl group to form an annulated oxazolidin-2-one which is N11-arylated by a substituted 4-aminophenyl ring (both in amorph or in any polymorph form), and pharmaceutically acceptable salts, prodrugs and solvates thereof. Preferably the phenyl ring has the structure as shown in formula I, and more preferred the para-position of benzene ring carries an optionally substituted amino group, such as a morpholino, thiomorpholino or piperazino substituent, said piperazino substituent may be further substituted at the second nitrogen atom. The para-position in the benzene ring might carry an amino group as part of an acylated indole system or the para-position in the benzene ring carries an acyl substituent as part of a lower alkanoyl system, or as part of an annulated pentanoyl or hexanoyl system tethered to an ortho position in the benzene ring. The benzene ring might be further substituted by either a fluorine or chlorine atom, or a methoxy group. The 2′-oxy and the 3′-N-demethylamino groups can be interconnected by a common carbonyl group to form an oxazolidin-2-one derivative, or the 3′-demethylamino group can carry a hydrogen atom or an additional C1-C6 alkyl group, which includes a methyl group, and the 2′-oxy group carries a hydrogen or a C1-C6 alkanoyl group. It is presently preferred that the Z-substituent in the 2-position of formula I is represented by hydrogen, fluorine or chlorine.

The compounds of the invention can be produced in manners known per se, i.e. in accordance with the methods disclosed in the above mentioned references which are hereby incorporated herein by references, or as described herein.

### DETAILED DISCLOSURE

In one embodiment, the invention relates to macrolides of Formula I (in both amorph or in any polymorph form), and pharmaceutically acceptable salts, prodrugs and solvates thereof,

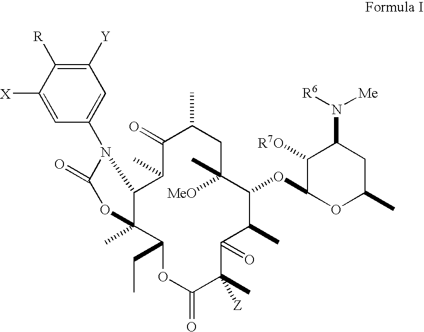

in which formula

X and Y independently represent hydrogen, hydroxy, cyano, carboxy, COOR¹³ (wherein R¹³ represents an optionally substituted aliphatic group), halogen, optionally substituted alkyl, optionally substituted alkoxy, or nitro, the benzene ring is optionally further substituted in the 2 and/or 6 position;

Z represents hydrogen or halogen;
R represents: (i) Morpholino, thiomorpholino, piperldino, or piperazino, said rings are optionally substituted on ring C atom(s) and/or ring N atom (such as with hydrogen, alkyl, alkoxy, —CO₂-alkyl, —CO-alkyl-OH, —CO-alkanediyl-OH, -alkanediyl-O-alkyl, -alkanedlyl-halogen, -alkenyl-O-alkyl, -alkenyl-halogen (any said groups being optionally substituted)); or
   (ii) optionally substituted alkanoyl;
   (iii) —N(R⁸)R⁹, wherein R⁸ and R⁹ independently represent hydrogen or optionally substituted alkyl; or R⁸ and R⁹ together represent the group -alkanediyl-R¹¹-alkanedlyl-, which group is optionally substituted, and R¹¹ represents CH₂, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl; or
   (iv) —N(R⁸)R⁹, wherein R⁸ and R⁹ independently represent hydrogen or optionally substituted alkyl; or R⁸ and R⁹ together represent the group -alkenyl-R¹¹-alkenyl-, which group is optionally substituted, and R¹¹ represents CH2, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl;
R represents, together with the vicinal X:
   (i) —CO-alkanediyl-, which is optionally substituted;
   (ii) —NR⁵-alkanedlyl-, which is optionally substituted and wherein R⁵ represents optionally substituted alkanoyl;
   (iii) —CO-alkenyl-, which is optionally substituted; or
   (v) —NR⁵-alkenyl-, which is optionally substituted and wherein R⁵ represents optionally substituted alkanoyl;

R⁶ represents hydrogen or optionally substituted alkyl; and R⁷ represents hydrogen, a hydroxy-protecting group or optionally substituted alkanoyl; or R⁶ and R⁷ together represent —CO—.

The invention also relates to a macrolide of Formula I, and pharmaceutically acceptable salts, prodrugs and solvates thereof, in which:

X and Y independently represent hydrogen, hydroxy, cyano, carboxy, COOR¹³ (wherein R¹³ represents an optionally substituted aliphatic group), halogen, optionally substituted alkyl, optionally substituted alkoxy, or nitro, the benzene ring is optionally further substituted in the 2 and/or 6 position;

Z represents hydrogen or halogen;
R represents: (i) Morpholino, thiomorpholino, piperidino, or piperazino, said rings are optionally substituted on ring C atom(s) and/or ring N atom (such as with hydrogen, alkyl, alkoxy, —CO₂-alkyl, —CO-alkyl-OH, -alkenyl-O-alkyl, -alkenyl-halogen (any said groups being optionally substituted)); or
   (ii) optionally substituted alkanoyl;
   (iii) —N(R⁸)R⁹, wherein R⁸ and R⁹ independently represent hydrogen or optionally substituted alkyl; or R⁸ and R⁹ together represent the group -alkenyl-R¹¹-alkenyl-, which group is optionally substituted, and R¹¹ represents CH2, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl; or
R represents, together with the vicinal X:
   (i) —CO-alkenyl-, which is optionally substituted; or
   (ii) —NR⁵-alkenyl-, which is optionally substituted and wherein R⁵ represents optionally substituted alkanoyl;

R⁶ represents hydrogen or optionally substituted alkyl; and R⁷ represents hydrogen, a hydroxy-protecting group or optionally substituted alkanoyl; or R⁶ and R⁷ together represent —CO—.

Presently preferred is macrolides wherein:

X and Y independently represent hydrogen, hydroxy, cyano, carboxy, COOR¹³ (wherein R¹³ represents C₁₋₆ alkyl), halogen, C₁₋₆ alkyl, C₁₋₆ alkoxy, or nitro;

Z represents hydrogen or halogen;
R represents: (i) Morpholino, thiomorpholino, piperidino, or piperazino (optionally substituted with halogen, C₁₋₆ alkyl, C₁₋₆ alkoxy, —CO₂—C₁₋₆-alkyl, —CO—C₁₋₆ alkanediyl-OH, —C₁₋₆ alkanediyl-O—C₁₋₆ alkyl, —C₁₋₆ alkanediyl-halogen; or
   (ii) C-₁₋₆ alkanoyl, optionally substituted with hydroxy;
   (iii) —N(R⁸)R⁹, wherein R⁸ and R⁹ independently represent hydrogen or C₁₋₆ alkyl; or R⁸ and R⁹ together represent the group —C₁₋₆ alkanediyl-R¹¹—C₁₋₆ alkanediyl-, and R¹¹ represents CH₂, S, O, SO, SO₂, SNR¹², S(O)NR¹² or NR¹², wherein R¹² represents hydrogen or C₁₋₆ alkyl (optionally substituted with halogen, hydroxy, C₁₋₆ alkoxy, amino, C₁₋₆ alkylamino, or di-C₁₋₆ alkylamino) or p-toluenesulfonyl; or
R represents, together with the vicinal X:
   (i) —CO—C₁₋₆-alkanediyl-, which is optionally substituted; or
   (ii) —NR⁵—C₁₋₆-alkanediyl-, wherein R⁵ represents C₁₋₆ alkanoyl;

R⁶ represents hydrogen or C₁₋₆ alkyl; and R⁷ represents hydrogen, a hydroxy-protecting group or C₁₋₆ alkanoyl; or R⁶ and R⁷ together represent —CO—.

Representative macrolides of the invention are the compounds of Formula I, in which formula X and Y are independently hydrogen, halogen or MeO. The halogen atom is preferably fluorine.

Z is hydrogen, fluorine or chlorine.

The R substituent is defined by the following options:

(i) R=Morpholino, thiomorpholino, or piperazino substituted at N-4 by R⁴.

These heterocyclic moieties further carry substituents R² and R³ which are independently defined by R² and R³═H, F, Cl, OMe.

The substituent at N-4 in the piperazino unit is defined by R⁴═H, CO₂Me, COCH₂OH, CH₂CH₂OMe, CH₂CH₂F.

(ii) R=Lower alkanoyl group substituted by at least one hydroxyl group.

(iii) When R and X are vicinal substituents they can be interconnected by R—X═—CO(CH₂)ₙ— where n=2, 3. The structures claimed are indanone and tetralone derivatives.

(iv) When R and X are vicinal substituents they can be interconnected by R—X═—NR⁵CH₂CH₂— where R⁵=lower alkanoyl substituted by at least one hydroxyl group, preferably —COCH₂OH.

The structures are indoline derivatives.

The substituents in the desosamine sugar part of the macrolide are defined by

R⁶═H or a lower alkyl group, preferably a methyl group.

R⁷═H or lower alkanoyl, preferably H.

R⁶ and R⁷ when taken together are defined by R⁶—R⁷═—CO—. The structures claimed are annulated oxazolidinone derivatives.

The invention also relates to processes for preparation of the macrolides of the invention, and novel intermediates that can be used in the processes. Processes for preparation of the macrolides of the invention or intermediates are disclosed in the specification and in the figures, and should all be considered to be embodiments of the present invention, both with the disclosed reactants and reaction details and with alternative reactants and/or reaction details.

In one embodiment, the invention relates to a process for the preparation of an 11-deoxy-11-aminomacrolide wherein the 11-amino and the 12-oxy groups are joined by a common carbonyl group to form an annulated oxazolidin-2-one structure which is N11-arylated by an optionally substituted phenyl ring, and/or pharmaceutically acceptable salts, prodrugs and/or solvates thereof, said process comprises reacting a 11-deoxy-11-aminomacrolide with a phenylisocyanate, which is optionally substituted on the phenyl ring. It is presently preferred that CuCl and/or NaH are be used as reagents, and/or the phenyl ring in the phenylisocyanate carries an electron withdrawing substituent, such as a halogen atom.

In an other embodiment, the invention relates to a process for the preparation of a compound of Formula I, pharmaceutically acceptable salts, prodrugs, polymorphs and/or solvates thereof,

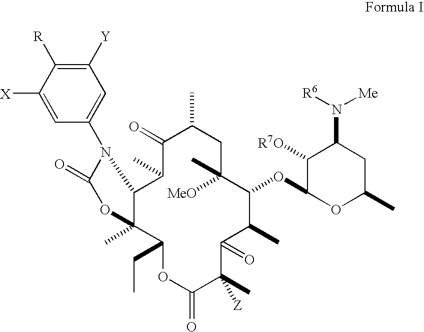

in which formula the substituents have the same meaning as above, said process comprises:
reacting an 11-deoxy-macrolide (such as a compound corresponding to formula I, but wherein there is a double bond between C10 and C11, the substituent at the 11 position is H, and the substituents at the 12 position are OH and methyl), with a 3-Y, 4-R, 5-X phenylisocyanate (wherein Y, R and X are defined as above) (such as depicted on FIG. 3); or
reacting an 11-deoxy-macrolide (such as a compound corresponding to formula I, but wherein there is a double bond between C10 and C11, the substituent at the 11 position is H, and the substituents at the 12 position are OH and methyl), with CDI and 3-Y, 4-R, 5-X aniline (wherein Y, R and X are defined as above) (such as depicted on FIG. 2); or
reacting a 11-amino-11-deoxy-11,12-urethane macrolide (such as compound corresponding to formula I, but wherein the N atom in the oxazolidinone ring is unsubstituted) with a 3-Y, 4-R, 5-X phenyl halide (such as depicted on FIG. 1);

and/or
deprotecting a protected derivative of a compound of formula I (e.g. a compound of formula I wherein R7 is a protecting group); and/or
converting a compound of formula I to another compound of formula I; and/or
reacting a compound of formula I with a pharmaceutically acceptable acid or base; and/or
crystallizing a compound of formula I in a suitable solvent.

The isocyanate reaction is greatly facilitated when the phenyl ring carries an electron withdrawing substituent, such as a halogen atom.

The processes of the invention are further disclosed in the following schemes, in which any substituent has the same meaning as above, unless otherwise defined in the context:

In Schemes 2-8 synthesis of a cyclic 11,12-urethane target compound 19 is presented. Structural analogues are prepared correspondingly. The initial substrate was clarithromycin (4) which was reacted with phosgene in order to effect demethylation and oxazolidinone annulation to the desosamine sugar as in structure 5. It has previously been established that the dimethylamino group in erythromycin derivatives will react with acid chlorides. The erythromycin products are the corresponding N-methylcarbamoyl derivatives after loss of a methyl group. Chloroformates are more commonly used. An early example is provided by a reaction of erythromycin A and benzyl chloroformate which yielded the corresponding N-benzyloxycarbonyl N-demethyl derivative in good yield.⁷ We reasoned that phosgene should provide a cyclic product, viz. an oxazolidinone derivative. The keto carbonyl group in unprotected clarithromycin (4) did not participate in the phosgene carbonylation reaction as shown in Scheme 2. Treatment with phosgene closed the hydroxyl groups at C-11 and C-12 to a cyclic carbonate. By increasing the reaction time and temperature, the C-2′ hydroxyl function and the 3′-dimethylamino group reacted to provide the cyclic 2′,3′-urethane in the desosamine sugar. Thus the dimethylamino-alcohol moiety of the desosamine sugar had suffered N-demethylation and was cyclised to the 2′,3′-carbamate 5. By this procedure we have discovered a carbonylation reaction with formation of a new and unexplored structure element for erythromycin A derivatives. The 2′,3′-urethane element has not previously been investigated for its influence on antibacterial activity. According to the literature, however, the 2′,3′-urethane element as a chemical structure was unexpectedly obtained from a side reaction in an NMR-study of epimerisation at C-2 in certain erythromycin derivatives.⁸ The other example available describes a total synthesis attempt of erythromycin antibiotics where cyclic 2′,3′-urethane was used for protection of the 2′-hydroxyl function and the 3′-dimethylamino group during macrolactonisation.⁹

The desosamine sugar has been considered essential for antibacterial activity. Acyclic 2′-carbamates were found to possess low antibacterial activity.¹⁰ No reference to cyclic 2′,3′-carbamate and antibacterial activity has been reported.

An allyl carbonate unit was formed at C-4″ in the cladinose sugar when allyl alcohol was added to the initially formed carbonyl chloride in the second step. Removal of the allyl carbonate at C-4″ in the initial product 5 could be effected in 93% yield by treatment with palladium acetate and triphenylphosphine as the catalyst system and triethylammonium formate as the reducing agent in aqueous ethanol under reflux. The deallylated product was obtained in 93% yield. In a modified protocol with Pd(dba)₂ and dppb as catalyst in the absence of a nucleophilic amine, the yield was lowered to 74%.

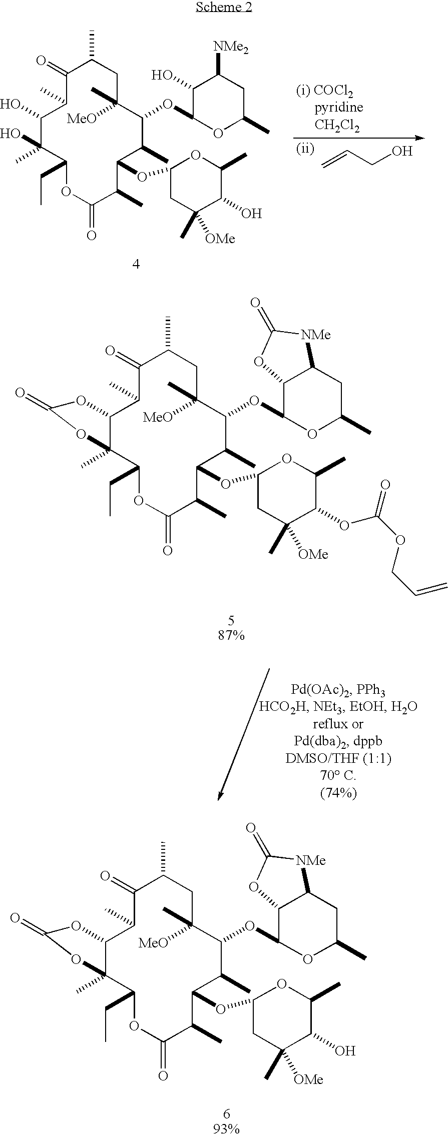

A three-step synthesis of the ketolide 8 is shown in Scheme 3. Initially the cladinose sugar was removed. The commonly employed procedures for cladinose removals involve hydrochloric acid in water or water-alcohol mixtures. Due to low solubility of the substrate 5 in such solvent systems, the conditions were modified with the use of trifluoroacetic acid in DMSO:water (9:1) which provided the 3-alcohol 7 in 63% yield. A subsequent oxidation of the alcohol 7 to the target ketolide 8 was performed in high yield by the Corey-Kim oxidation protocols.^{2,11}Together with a modified version of the Pfitzner-Moffat oxidation, the Corey-Kim procedure constitutes the most popular method for oxidation of the hydroxyl function at C-3 in erythromycin A derivatives.¹²

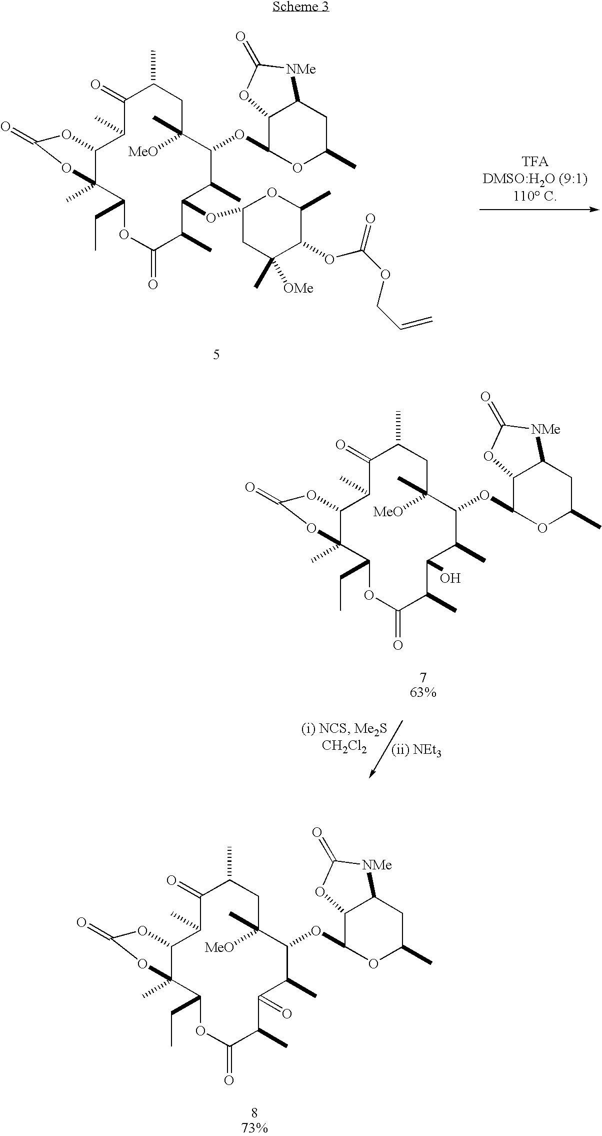

Alternatively, the order of the reactions can be changed. The cladinose sugar could be removed first, followed by carbonylation and oxidation. The strategy is shown in Scheme 4. Descladinosylclarithromycin (9) was available by hydrolytic removal of the cladinose sugar.¹³ When compound 9 was subjected to phosgene carbonylation with allyl alcohol as quenching agent, the C-3 allyl carbonate 10 was formed. The reaction was slower and less clean than for the corresponding macrolide substrate 4 with the cladinose sugar intact. The chemical yield was reduced to 41%. This behavior is explained by a lower access to the C-3 hydroxyl function in 9 compared to the hydroxyl function at C-4″ in the cladinose sugar. Formation of the C-3 chloroformate intermediate is therefore slower.

The substrate 10 was almost insoluble in solvents usually employed in Pd-catalysed removals of allyl carbonates. Starting from the protocol reported by Genet et al.,¹⁴ conditions were modified to fit substrate 10. The original conditions include Pd(dba)₂ and dppe or dppb as catalyst systems. Diethylamine was used as nucleophile. The reaction was performed at ambient temperature in a THF solution. By changing the solvent to DMSO:THF (1:1) and increasing the temperature to 70° C., dissolution of 10 occurred. Removal of the allyl carbonate then proceeded smoothly when Pd(dba)₂, dppb and diethylamine were added. Later, it was found that the reaction proceeded equally well in the absence of diethylamine.

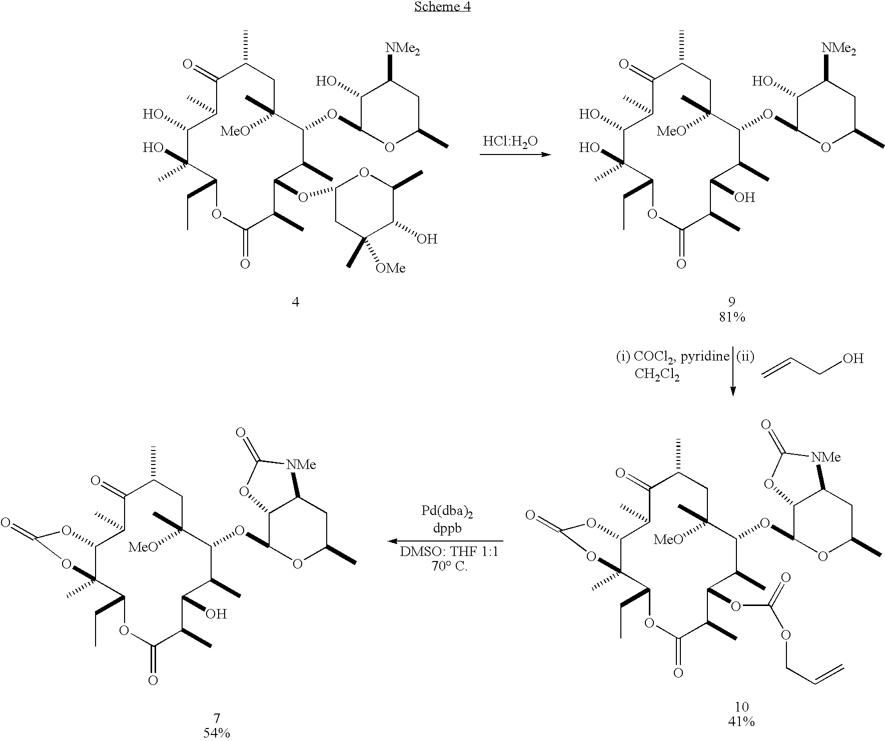

Addition of nucleophiles to the carbonylated species 5 is a synthetically useful reaction as shown in Scheme 5. Sodium azide or lithium propanethiolate was used to effect removal of the cyclic carbonate as well as the 4″-allyl carbonate moieties. A double bond was concurrently introduced into the 10,11-positions. The double bond is conjugated to the keto carbonyl group. The urethane moiety was left untouched. In contrast, non-cyclic carbamate protecting groups are removed by lithium propanethiolate.¹⁵ The reaction with sodium azide required the use of elevated temperatures, the lithium propanethiolate reaction proceeded at ambient temperature. Commonly, the 10,11-double bond in erythromycin macrolides is introduced via base (DBU) mediated elimination of the 11,12-carbonate,¹⁶ or the 11-mesylate.¹³

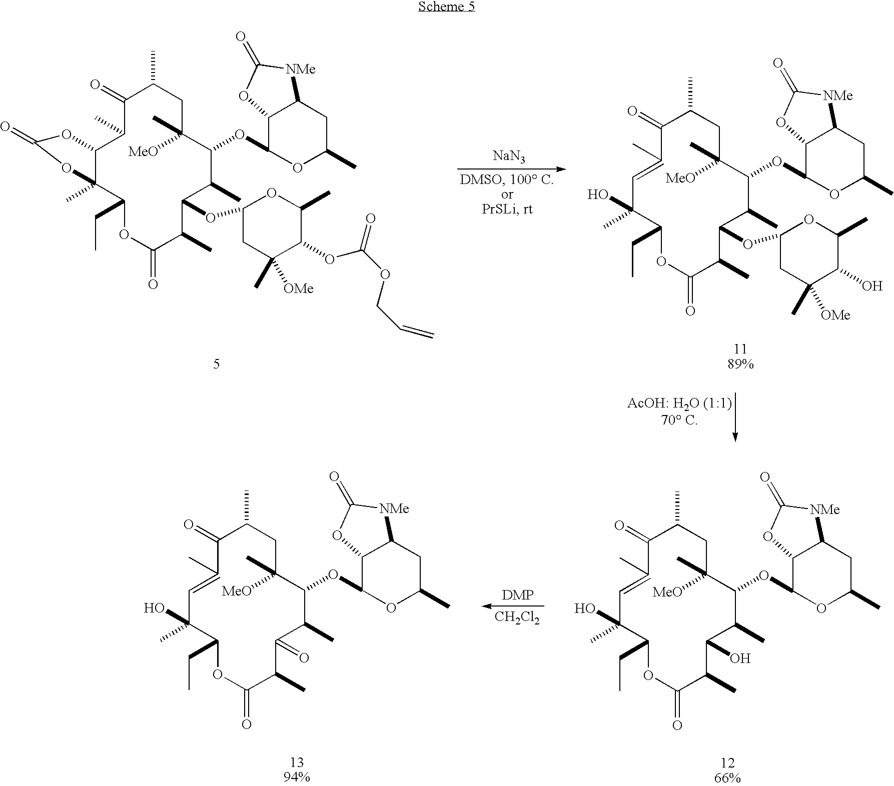

Hydrolytic removal of cladinose was effected under mild conditions, viz. by the use of aqueous acetic acid at 70° C. when the 3-hydroxy derivative 12 was formed in 66% yield. Mild acid conditions are employed to avoid rearrangement processes involving the lactone ring taking place under more strongly acidic conditions. The target keto lactone 13 was obtained in 94% yield when the alcohol 12 was subjected to the Dess-Martin periodinane conditions.¹⁷ These conditions are in general very good for ketolide formation. The product 13 was obtained in an almost pure form after alkaline extraction.

In the first of the two commonly used methods as indicated in Scheme 6 for the introduction of the 11,12-carbamate structure, 1,1′-carbonyidiimidazole (CDI) is reacted with the 12-OH group in the macrolide to form an O-acylimidazolyl intermediate 14 which is subsequently treated with a primary amine. The initial carbamate is subsequently cyclised by addition of the amido nitrogen onto the C—C double bond in a Michael fashion to form the oxazolidinone product 16. Alternatively, ammonia is used for production of the N-unsubstituted cyclic 11,12-urethane 15. The latter can be N-substituted by alkylation reactions under basic conditions or by metal-catalysed cross-coupling reactions to furnish target compound 16. The absolute configurations at C-10 and C-11 as in clarithromycin are lost when the 10,11-double bond is introduced in the macrolide. The subsequent intramolecular Michael addition dictates the stereochemical outcome for the two stereocenters at C-10 and C-11. Conformational restriction of the macrolide ring directs the carbamate attack at C-11 from the same side as the C-12 substituent, leading to the natural (11R)-configuration. Subsequently, the stereochemical outcome at C-10 is decided by the protonation of the intermediate enolate. Mixtures may be obtained with the natural (10R)-configuration predominating.¹⁶ When bulky amines are introduced, the natural (10R)-isomer is isolated as the only product. The opposite (10S)-configuration renders the 11,12-carbamates virtually biologically inactive.¹³

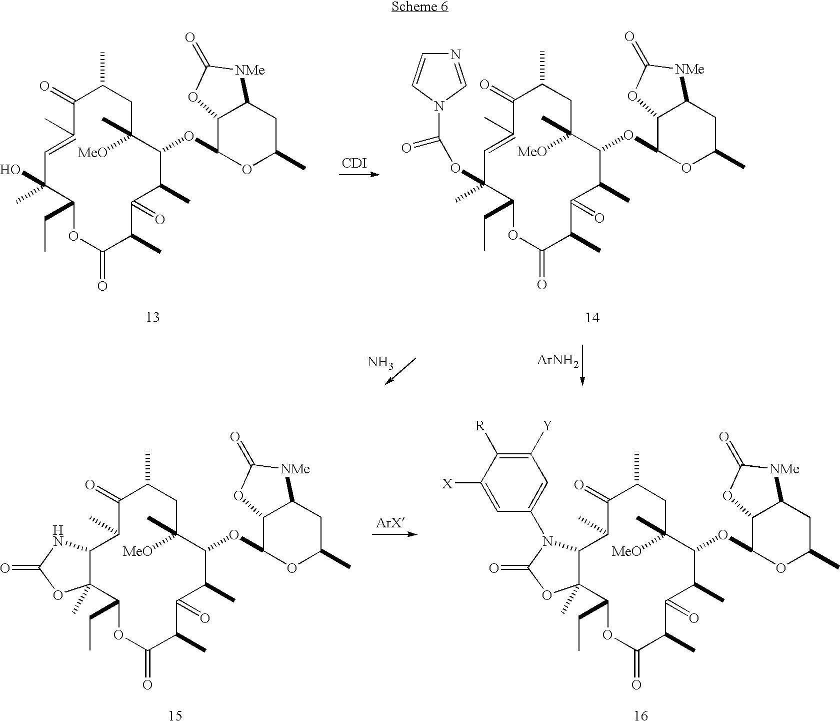

In the second approach, the C-12 alcohol is reacted with an isocyanate, leading to the same carbamate intermediate as described in the previous case. A subsequent ring-closing reaction gives the oxazolidinone structure. The reagents for the latter approach in this work were 4-bromophenylisocyanate, sodium hydride and copper(I) chloride. The 4-bromophenyl carbamate 17 was obtained in 62% yield. TLC indicated full conversion after about two hours, but the NMR spectra indicated mixtures of stereoisomeric products. Stirring for a longer time resulted in isomerisations providing a single stereomer. The role of the copper(I) chloride is to form a complex with the isocyanate to facilitate the first reaction step. The bromine in the product was introduced in order to have a versatile substrate for further phenyl substitutions by cross-coupling reactions as in carbylations and introduction of hetero-substituents exemplified by amino groups. Reference is made to the recently published procedures for amination of aryl halides effected by Pd-catalysed reactions.¹⁸⁻²¹ Members of the latter group can also be prepared directly using corresponding amino isocyanates as reactants. An example is given by the

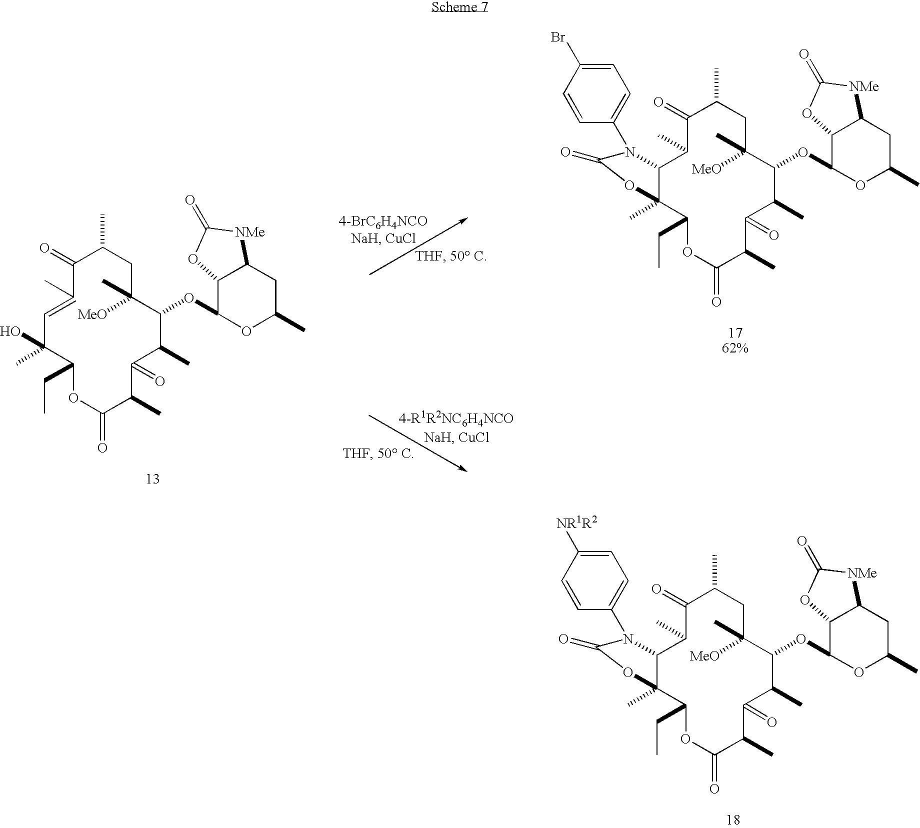

preparation of the amino derivative 18 in Scheme 7. In an alternative approach the amino nitrogen in the benzene 4-postion can initially be introduced via a higher oxidation state nitrogen followed by a reduction to the amino function. The concept is indicated by the use of p-nitrophenylisocyanate in the reaction with substrate 13 to furnish cyclic products 18 where NR¹R²═NO₂. The macrolide structures can be prepared by the isocyanate pathway.

The target compound 19 in Scheme 8 was available by coupling of the enone 13 with 3-fluoro-4-morpholinophenylisocyanate. The stereochemistry at C-10 and C-11 was as in the natural product since no coupling was observed between the protons in the two positions. H-11 appeared as a singlet in the ¹H NMR spectrum. From the literature it is known that the coupling constant J_{H10,H11} is close to zero for the natural configuration at C-10 and C-11 in macrolides.¹⁵

Hence the product 19 has been assigned the natural macrolide configuration. The isocyanate side-chain can be prepared in a separate and well established reaction sequence as reported for the linezolid synthesis.^{22,23} Its further insertion reaction into substrate 13 provided the target compound 19 in 56% yield.

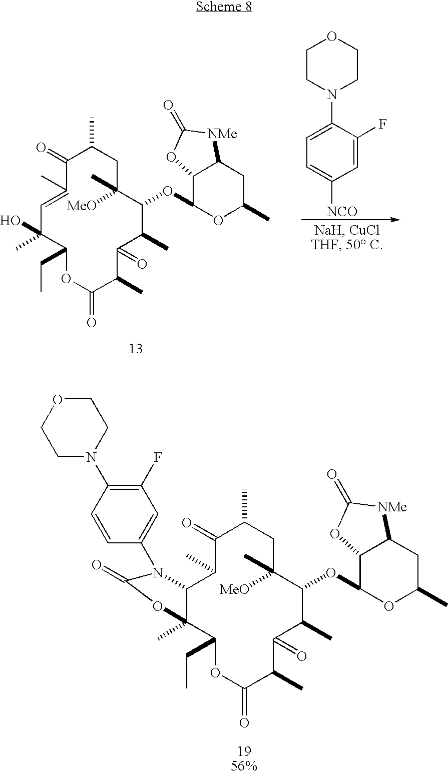

The subsequent work illustrates a process for the preparation of the compounds in Scheme 9 as members of the group where the amino nitrogen in desosamine is substituted by one or two alkyl groups. A suitable substrate for the overall process is the ketolide 20 in Scheme 9. The first reaction step involves mono-demethylation of the dimethylamino group in the substrate 20 by analogy to photolytical or thermal demethylation reactions of erythromycin and its derivatives with molecular iodine in the presence of a base such as NaOAc.²⁴ Selective monodemethylation by means of N-iodosuccinimide (NIS) is claimed to be a superior method.²⁵ Alternatively,

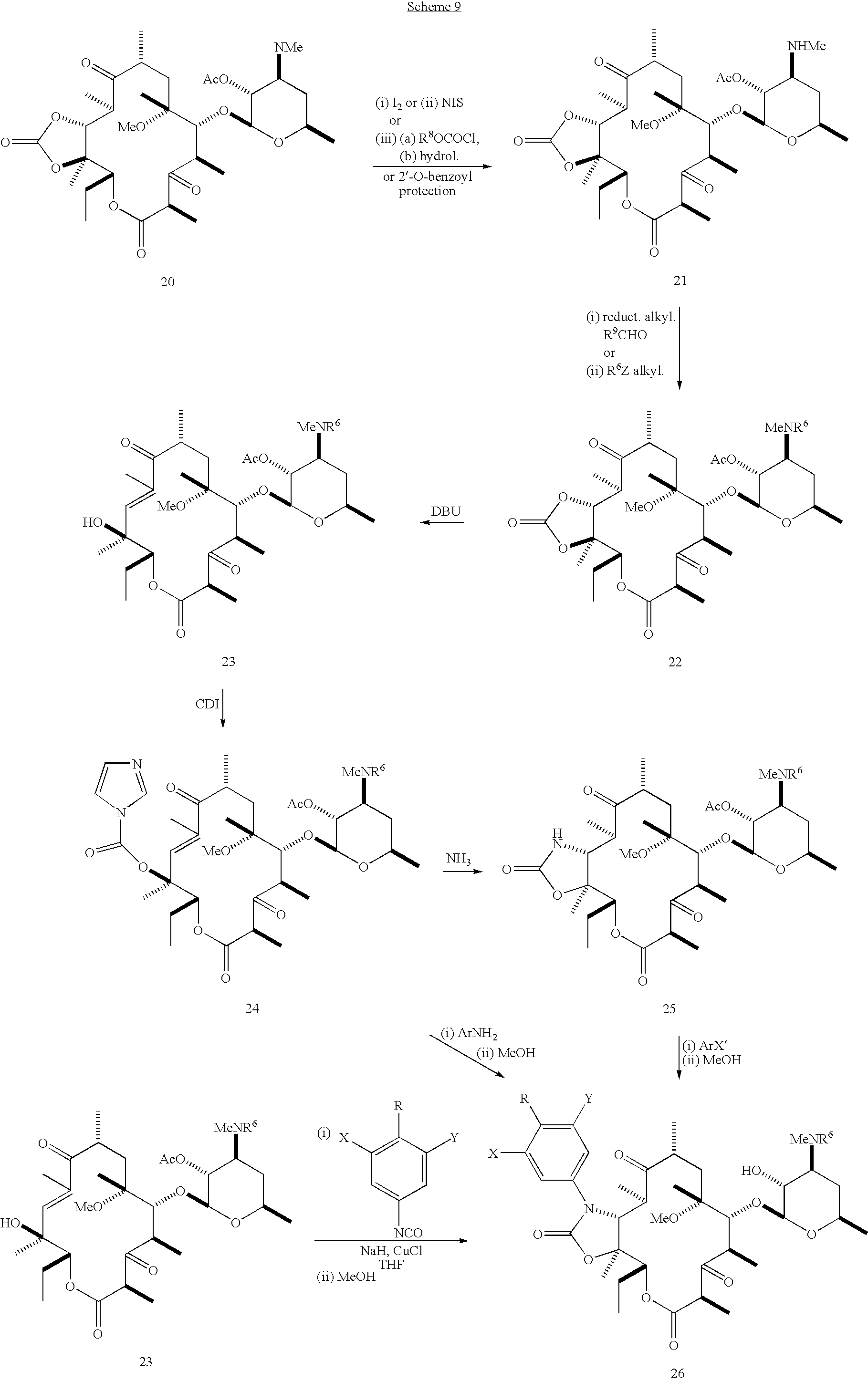

chloroformates are more commonly used. The initial product in that case is a urethane of the monodemethylated substrate. With benzyl chloroformate, the N-benzyloxycarbonyl N-demethyl product on hydrogenolysis will liberate the free amine.⁷ 1-Chloroethyl chloroformate is the preferable reagent for N-demethylation. The initial product is the N-demethyl N-1-chloroethyl carbamate which in methanol undergoes methanolysis providing the amine hydrochloride.²⁶ The monomethylamine derivative is subsequently alkylated either by reductive alkylation procedures from the appropriate aldehyde, or by a simple direct N-alkylation to provide the unsymmetrical dialkylamine 22. The cyclic carbonate moiety is cleaved under basic conditions, preferably by DBU, to provide the conjugated enone 23. With carbonyidiimidazole the initial product is the 12-O-acylimidazolide 24 which reacts with a primary amine to form the target compound 26. Alternatively, the imidazolide 24 reacts with ammonia or equivalents to form the secondary amide 25 which is to be substituted at the nitrogen by an aryl function to provide the target molecule 26. In a more direct procedure, the conjugated enone 23 is reacted with the appropriate isocyanate to form the target molecule 26 in either a two-step or an one-pot reaction. The 2′-protecting group might be removed using appropriate methods.

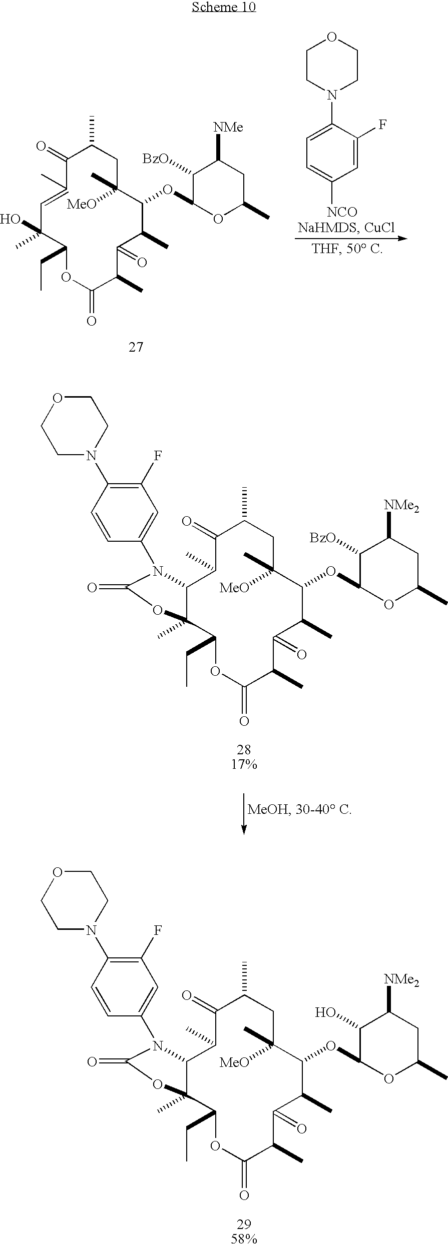

The cyclic 11,12-carbamate 29 was prepared in two steps from the 2′-O-benzoyl protected enone 27 (prepared according to literature procedures^{27,28}) Reaction of the substrate (27) with 3-fluoro-4-morpholinophenyl isocyanate in the presence of copper(I) chloride and sodium bis(trimethylsilyl)amide, NaHMDS, afforded the N-substituted 11,12-carbamate 28 in 17% yield after purification. Removal of the 2′-O-benzoyl group, with formation of the free hydroxy function, was accomplished by stirring in methanol for 4 days at elevated temperatures.

The invention also relates to pharmaceutical compositions comprising a compound according to the invention, together with a pharmaceutically acceptable carrier or excipient, and the use of a compound according to the invention for manufacture of pharmaceutical composition, such as an antibacterial composition.

Further, the invention relates to a method for treatment of an animal (such as a mammal, including a human), said method comprising administering a pharmaceutical composition (or a compound) according to the invention, to the animal.

### THE DRAWINGS

FIG. 1 illustrates the process for preparation of a compound of the invention by reacting the corresponding 11,12 urethane derivative with a substituted phenyl halide, i.e. X′ in the formula represents halogen. The reaction can be carried out under basic conditions and/or by metal-catalysed cross-coupling.

FIG. 2 illustrates the process for preparation of a compound of the invention by reacting the corresponding 11-deoxy macrolide with a substituted aniline.

FIG. 3 illustrates the process for preparation of a compound of the invention by reaction the corresponding 11-deoxy macrolide with a substituted phenyl isocyanide. In this process, NaH and/or CuCl can be used as reagent(s) and THF can be used as solvent.

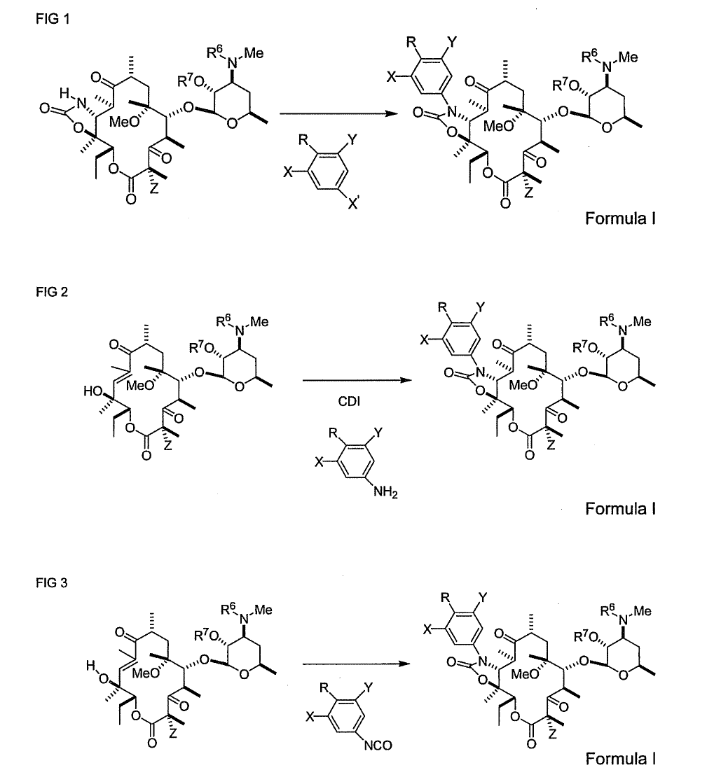

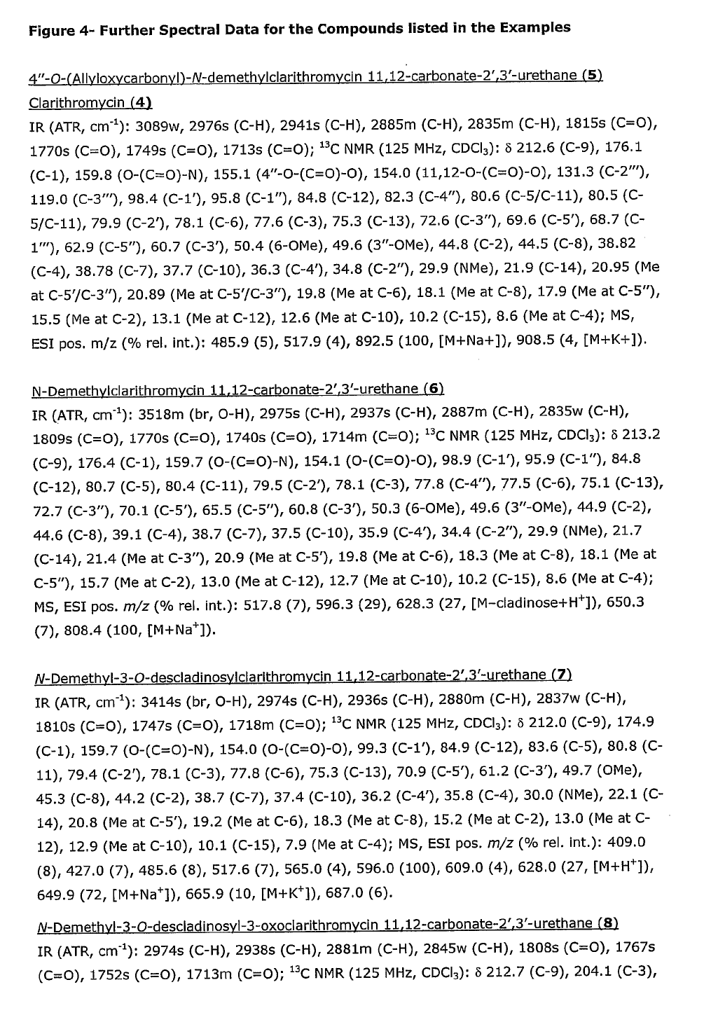

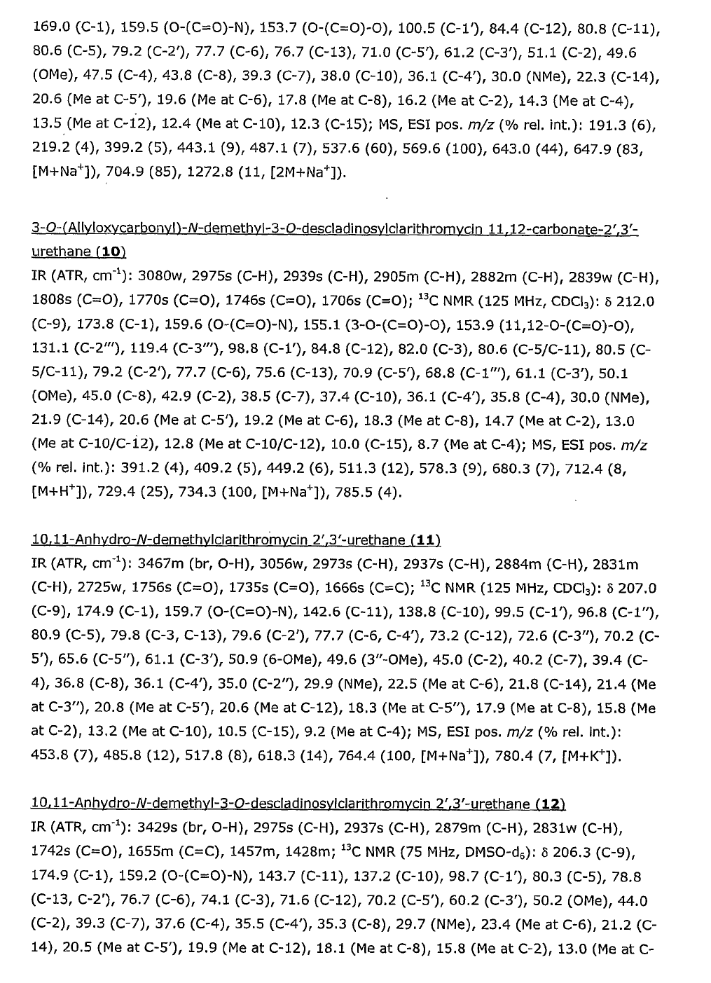

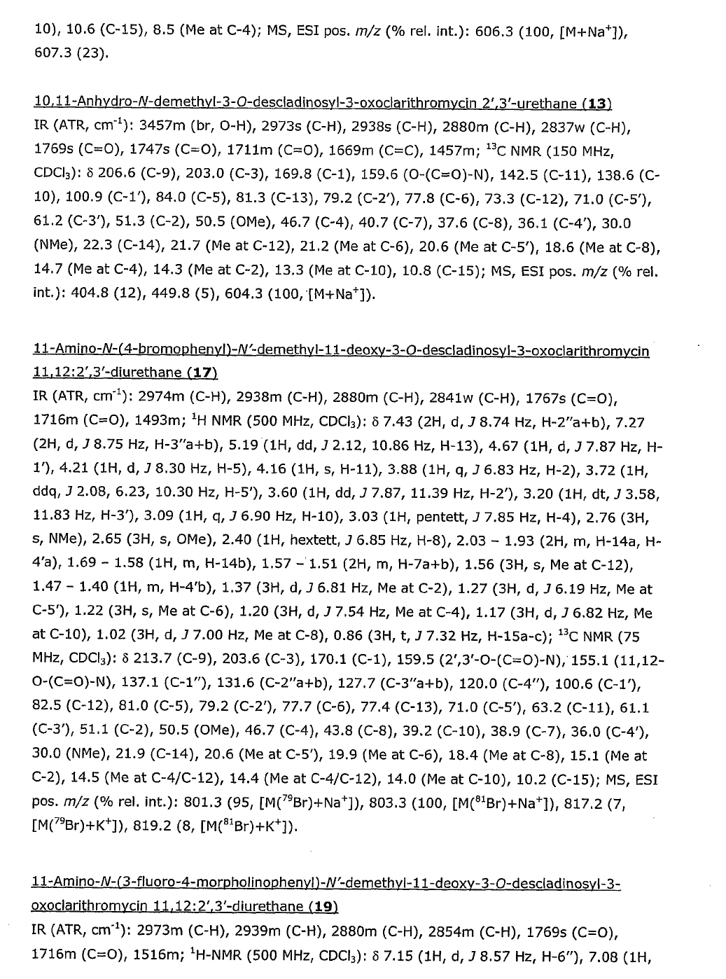

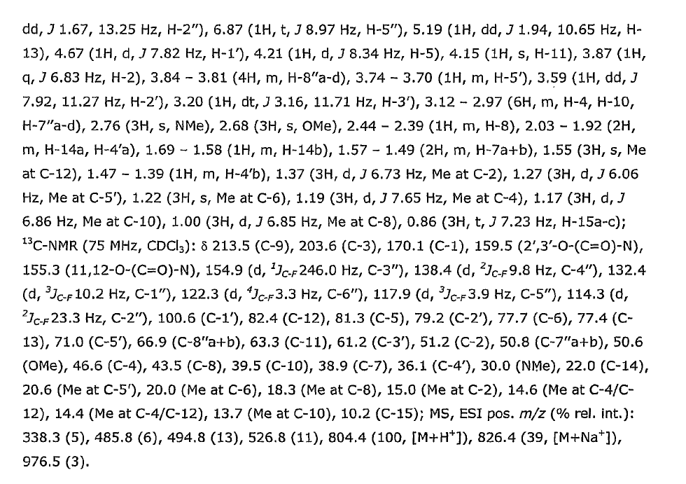

In any of the depicted processes, a compound wherein R7 represents H can be obtained by reacting a compound wherein R7 represents Ac with an alkanol, e.g. methanol. The substituents in the formulas have the same meaning as in claim 1.

Definitions

In the formulas, the substituents have the same meanings as in IUPAC Compendium of Chemical Terminology unless otherwise defined. When the substituent definition comprises a range (e.g. C1-C6 or C1 to C10), the range is understood to comprise all integers in that range, i.e. 1, 2, 3, 4, 5, 6, 7, 8, 9, 10, 11, 12, 13, 14, 15, 16, 17, 18, 19, 20, 21, 22 etc.

The term “substituted” means that one or more (such as 1, 2, 3, 4, 5, or 6) hydrogen atoms are substituted with substituents independently selected from groups such as: halogen atoms, nitro groups, hydroxyl, mercapto, cyano, carbamoyl, optionally substituted amino, optionally substituted alkyl (e.g. perhalogenalkyl), optionally substituted alkenyl, optionally substituted alkynyl, optionally substituted cycloalk(en/yn)yl, optionally substituted aryl, optionally substituted alkoxycarbonyl, optionally substituted aryloxycarbonyl, optionally substituted alkoxy, optionally substituted alkylthio, optionally substituted (hetero)aryl, optionally substituted (hetero)aryloxy or acyl groups. Two hydrogen atoms on the same carbon atom can be substituted with a divalent substituent, such as optionally substituted C1-C6 alkylene, O, NH, S.

The term “halogen” (or “halide”) represents fluoro, chloro, bromo, or iodo.

The term “heteroatom” or “hetero” includes atoms such as O, S, or N.

The term “alkyl” includes straight or branched chain aliphatic hydrocarbon groups that are saturated and have 1 to 15 carbon atoms. Preferably, the alkyl group has 1-10 carbon atoms, and most preferred 1, 2, 3, 4, 5, or 6 carbon atoms. The alkyl groups may be interrupted by one or more heteroatoms, and may be substituted, e.g. with groups as defined above, such as halogen, hydroxyl, aryl, cycloalkyl, aryloxy, or alkoxy. Preferred straight or branched alkyl groups include methyl, ethyl, propyl, isopropyl, butyl and t-butyl. The term “alkoxy” stands for an —O-alkyl group.

The term “cycloalkyl” includes straight or branched chain, saturated or unsaturated aliphatic hydrocarbon groups which connect to form one or more rings of preferably 3, 4, 5, 6, or 7 ring members, which can be fused or isolated. The rings may be substituted, e.g. with groups as defined above, such as halogen, hydroxyl, aryl, aryloxy, alkoxy, or alkyl. Preferred cycloalkyl groups include cyclopropyl, cyclobutyl, cyclopentyl and cyclohexyl.

The term “alkenyl” includes straight or branched chain hydrocarbon groups having 2 to 15 carbon atoms (e.g. 2, 3, 4, 5, 6 or 10 carbon atoms) with at least one carbon-carbon double bond, the chain being optionally interrupted by one or more heteroatoms. The chain hydrogens may be substituted, e.g. with groups as defined above, such as halogen. Preferred straight or branched alkenyl groups include vinyl, allyl, 1-butenyl, 1-methyl propenyl and 4-pentenyl.

The term “alkylene” represents an alkanediyl group commonly but not necessarily having the free valencies on adjacent carbon atoms. E.g. —CH(CH₃)CH₂— propylene. As an illustration, the group —CO-alkanediyl-OH comprises e.g. the group —CO—(CH₂)ₙ—OH, wherein n can be an integer between 1 and 6.

The term “alkynyl” includes straight or branched chain hydrocarbon groups having 2 to 15 carbon atoms (e.g. 2, 3, 4, 5, 6 or 10 carbon atoms) with at least one carbon-carbon triple bond, the chain being optionally interrupted by one or more heteroatoms. The chain hydrogens may be substituted, e.g with groups as defined above, such as halogen. Preferred straight or branched alkynyl groups include ethynyl, propynyl, 1-butynyl, and 4-pentynyl.

The term “cycloalkenyl” includes straight or branched chain, saturated or unsaturated aliphatic hydrocarbon groups which connect to form one or more non-aromatic rings of preferably 3, 4, 5, 6, or 7 ring members containing a carbon-carbon double bond, which can be fused or isolated. The rings may be substituted, e.g. with groups as defined above, such as halogen, hydroxyl, alkoxy, or alkyl. Preferred cycloalkenyl groups include cyclopentenyl and cyclohexenyl.

The term “aryl” refers to carbon-based rings which are aromatic. The rings may be isolated, such as phenyl, or fused, such as naphthyl. The ring hydrogens may be substituted, e.g. with groups as defined above, such as alkyl, halogen, free or functionalized hydroxy, trihalomethyl, etc. Preferred aryl groups include phenyl, 3-(trifluoromethyl)phenyl, 3-chlorophenyl, 3-fluoro-4-morpholinophenyl, and 4-fluorophenyl.

The term “heteroaryl” refers to aromatic hydrocarbon rings (having such as 3, 4, 5, 6, or 7 ring members) which contain at least one (e.g. 1, 2, 3, 4, or 5) heteroatom(s) in the ring. Heteroaryl rings may be isolated, preferably with 5 to 6 ring atoms, or fused, preferably with 8, 9 or 10 ring atoms. The heteroaryl ring(s) hydrogens or heteroatoms with open valency may be substituted, e.g. with groups as defined above, such as alkyl or halogen. Examples of heteroaryl groups include imidazole, pyridine, indole, quinoline, furan, thiophene, pyrrole, tetrahydroquinoline, dihydrobenzofuran, and dihydrobenzindole.

The term “aliphatic group” comprises both saturated and unsaturated, straight chain (i.e., unbranched), branched, cyclic, or polycyclic aliphatic hydrocarbons, which are optionally substituted with one or more functional groups. The term includes, but is not limited to, alkyl, alkenyl, alkynyl, cycloalkyl, cycloalkenyl, and cycloalkynyl moieties. It is presently preferred that alkyl or other aliphatic groups have 1-6 carbon atoms (which may be substituted or unsubstituted as specified). For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl.

The term “heteroaliphatic group” refers to aliphatic moieties (cf. the term aliphatic as defined above), which contain one or more oxygen, sulfur, nitrogen, phosphorous or silicon atoms, e.g., in place of carbon atoms. Heteroaliphatic moieties may be substituted or unsubstituted, branched, unbranched, cyclic or acyclic, and include saturated and unsaturated heterocycles such as morpholino, pyrrolidinyl, etc.

The term “carbocyclic group/ring” includes a mono or bicyclic carbocyclic ring (e.g., cycloalkyl or cycloalkenyl groups such as cyclopropyl, cyclobutyl, cyclopentyl, cyclohexyl, cycloheptyl, cyclooctyl, cyclononyl, cyclopentenyl, cyclohexenyl, and bicyclo[2.2.1]heptanyl, bicyclo[3.2.1]octanyl and bicyclo[5.2.0]nonanyl, etc.); optionally containing 1-2 double bonds and optionally substituted by 1 to 3 suitable substituents as defined above.

The term “heterocyclic group/ring” includes both heteroaryl as above defined as well as non-aromatic ring systems having five to fourteen members, preferably five to ten, in which one or more ring carbons, preferably one to four, are each replaced by a heteroatom such as N, O, or S. Examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocyclic” whether saturated or partially unsaturated, also refers to rings that are optionally substituted with substituents as above defined.

The term “acyl” encompasses carboxylic acyl groups having the formula A-C(═O)—, in which formula A represents a substituent as defined above, such as an alkyl, alkenyl, aryl, heteroaryl or aralkyl group, the chain in said groups being optionally interrupted by one or more heteroatoms and the groups being optionally substituted, e.g. by one or more substituents as defined above. Examples on acyl groups are formyl, C1-C6 alk(en/yn)ylcarbonyl, arylcarbonyl, aryl-C1-C6 alk(en/yn)ylcarbonyl, cycloalkylcarbonyl, or cycloalkyl-C1-C6 alk(en/yn)ylcarbonyl group. Also, the term acyl comprises any of the above groups in which the C(═O) group is replaced by C(αS) or C(N—R), R is H or a substituent as defined above.

The term “hydroxy-protecting group” is intended to mean any group used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. Alkoxyalkyl protecting groups are groups such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals.

A “protected hydroxy” group is a hydroxy group derivatised or protected by any of the above groups commonly used for the temporary or permanent protection of hydroxy functions, e.g. the silyl, alkoxyalkyl, acyl or alkoxycarbonyl groups, as previously defined.

The term “solvate” represents an aggregate that comprises one or more molecules of the compound of the invention, with one or more molecules of solvent. Solvents may be, by way of example, water, ethanol, acetone, THF, DMA, or DMF. It should be understood that solvates (e.g., hydrates) of the compounds of the present invention are also within the scope of the present invention. Methods of solvation are generally known in the art.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates. In addition, zwitterions (“inner salts”) may be formed from the compounds of the present invention.

The term “prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the present invention. The term “prodrug” refers to compounds that are rapidly transformed in vivo to yield the parent compound of the above formula, for example by hydrolysis in blood. A thorough discussions is provided in T. Higuchi and V. Stella, “Pro-drugs as Novel Delivery Systems, Vol. 14 of the ACS Symposium Series, and in Edward B. Roche, ed., “Bioreversible Carriers in Drug Design”, American Pharmaceutical Association and Pergamon Press, 1987, both of which are incorporated herein by reference, and comprises pharmaceutically acceptable esters of the macrolides of the invention.

Various forms of prodrugs are well known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krosgaard-Larsen and H. Bundgaard, Chapter 5, “Design and Application of Prodrugs,” by H. Bundgaard, p. 113-191 (1991);

c) H. Bundgaard, et al., Advanced Drug Delivery Reviews, 8, 1-38 (1992);

d) H. Bundgaard, et al., Journal of Pharmaceutical Sciences, 77, 285 (1998); and

e) N. Kakeya, et al., Chem Phar Bull, 32, 692 (1984).

The pharmaceutical compositions of the present invention comprise a therapeutically effective amount of a compound of the present invention formulated together with one or more pharmaceutically acceptable carriers or excipients. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate. Coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value failing within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### 4″-O-(Allyloxycarbonyl)-N-demethylclarithromycin 11,12-carbonate-2′,3′-urethane (5)

Clarithromycin (4, 5.00 g, 6.69 mmol) was dissolved in dichloromethane (100 ml), and pyridine (6.30 ml, 77.9 mmol) and phosgene (20% in toluene, 20.0 ml, 38.0 mmol) were added. The reaction mixture was stirred at rt for 5 h. Allyl alcohol (9.00 ml, 132 mmol) was added, and the stirring was continued for another 30 min (yellow solution). Aqueous sodium hydroxide was added, and the product was extracted with dichloromethane. The combined organic layers were washed with water and brine, dried over magnesium sulfate and filtered. Evaporation of the filtrate and chasing with toluene provided a pale yellow solid which was recrystrallised from toluene; yield 5.07 g (87%) of the allyl carbonate 5 as a white solid; mp.: 307-310° C. (toluene). (Calc. for C₄₃H₆₇NO₁₇: C, 59.36; H, 7.76. Found: C, 60.01; H, 7.36%). HRMS, ESI pos.: Calc. for M+Na⁺=C₄₃H₆₇NNaO₁₇: 892.4301. Found: 892.4327. See FIG. 4 for further Spectral Data.

### N-Demethylclarithromycin 11,12-carbonate-2′,3′-urethane (6)

Method 1: A suspension of the substrate 5 (300 mg, 0.35 mmol), triethylamine (0.17 ml, 1.2 mmol), formic acid (0.040 ml, 1.1 mmol), palladium acetate (5 mg, 0.022 mmol, 6 mol %) and triphenylphosphine (22 mg, 0.080 mmol) in 80% aqueous ethanol (6 ml), was refluxed for 1.5 h. The resulting yellow mixture was cooled to room temperature, evaporated to dryness and the residual material purified by flash chromatography on silica gel (25 g) using ethyl acetate:triethylamine 98:2; yield 251 mg (93%) of the deprotected compound 6 as a white solid. Recrystallisation from isopropanol removed the last traces of triphenylphosphine. Method 2: The substrate 5 (103 mg, 0.12 mmol) was dissolved in THF:DMSO (1:1, 2.6 ml) at 70° C. 1,4-Bis(diphenylphosphino)butane (dppb, 8 mg, 0.019 mmol) and bis(dibenzylidene-acetone)palladium (Pd(dba)₂, 10 mg, 0.017 mmol, 14 mol %) were added, and the reaction mixture was stirred at 70° C. for 4 h 15 min. The mixture was cooled to room temperature, aqueous sodium hydroxide added, and the mixture extracted with dichloromethane and the combined organic layers washed with water and brine. The combined organic extracts were dried over magnesium sulfate, filtered, the filtrate evaporated at reduced pressure and the residual material subjected to flash chromatography on silica gel (10 g) using hexane:ethyl acetate:triethylamine 23:75:2; yield 69 mg (74%) of 6 as a white solid; mp. 292-295° C. (dec.) (isopropanol). (Calc. for C₃₉H₆₃NO₁₅: C, 59.60; H, 8.08. Found: C, 58.91; H, 7.89%). HRMS, ESI pos.: Calc. for M+Na⁺═C₃₉H₆₃NNaO₁₅: 808.4089. Found: 808.4111. See FIG. 4 for further Spectral Data.

### N-Demethyl-3-O-descladinosylclarithromycin 11,12-carbonate-2′,3′-urethane (7)

Trifluoroacetic acid (0.60 ml, 7.8 mmol) was added to a stirred suspension of the substrate 5 (1.00 g, 1.15 mmol) in DMSO:water (9:1, 50 ml) at 110° C. All material had dissolved after stirring for 4 h 15 min. TLC indicated full conversion after 6 h. The mixture was cooled to room temperature, aqueous sodium hydroxide was added, and the mixture was extracted with dichloromethane. The combined organic layers were washed with water and brine, dried over magnesium sulfate, filtered, the filtrate evaporated and the residual material subjected to flash chromatography on silica gel (80 g) using dichloromethane:isopropanol:triethylamine 97:1:2 followed by recrystallisation from dichloromethane/toluene; yield 456 mg (63%) of the product 7 as a white solid; mp. 294° C. (dec.) (dichloromethane:toluene). (Calc. for C₃₁H₄₉NO₁₂: C, 59.31; H, 7.87. Found: C, 58.71; H; 7.64%). HRMS, ESI pos.: Calc. for M+H⁺═C₃₁H₅₀NO₁₂: 628.3327. Found: 628.3347. See FIG. 4 for further Spectral Data.

### N-Demethyl-3-O-descladinosyl-3-oxoclarithromycin 11,12-carbonate-2′,3′-urethane (8)

N-Chlorosuccinimide (NCS, 59 mg, 0.44 mmol) was dissolved in dichloromethane (3 ml) and cooled to −16° C. Dimethyl sulfide (0.037 ml, 0.50 mmol) was added dropwise over 5 min, and the mixture was stirred for another 10 min. A solution of the alcohol 7 (170 mg, 0.27 mmol) in dichloromethane (20 ml) was added dropwise over 30 min, while the temperature was kept between −16 and −10° C. The reaction mixture was stirred for 1.5 h, while the temperature was allowed to reach −5° C. Triethylamine (0.041 ml, 0.29 mmol) was added dropwise over 5 min. The mixture was stirred for 1.5 h at −5° C., allowed to reach room temperature (colourless solution), aqueous sodium hydroxide added, and the product extracted with dichloromethane. The combined organic layers were washed with water and brine, dried over magnesium sulfate and filtered. Evaporation of the filtrate furnished 149 mg of a white solid. Purification by flash chromatography on silica gel (12 g) using dichloromethane:triethylamine 98.5:1.5 provided 124 mg (73%) of the target ketolide 8 as a white solid; mp. 283-285° C. (dec.) (dichloromethane:hexane). (Calc. for C₃₁H₄₇NO₁₂: C, 59.51; H, 7.57. Found: C, 58.55; H, 7.45%). HRMS, ESI pos.: Calc. for M+Na⁺═C₃₁H₄₇NNaO₁₂: 648.2990. Found: 648.3019. See FIG. 4 for further Spectral Data.

### 3-O-Descladinosylclarithromycin (9)¹³

Clarithromycin (4, 1.25 g, 1.67 mmol) was added in portions to 1.0 M aqueous hydrochloric acid (30 ml, 30 mmol), and the mixture stirred at ambient temperature for 30 min when all the solid had gone into solution. The reaction had gone to completion after 2 h. Aqueous sodium hydroxide was added, the mixture extracted with ethyl acetate, the combined organic extracts washed with water and brine, dried over magnesium sulfate, filtered, the filtrate evaporated and the residual material subjected to flash chromatography on silica gel (50 g) using ethyl acetate:triethylamine 96:4; yield 802 mg (81%) of the product 9 as a white foam. HRMS, ESI pos.: Calc. for M+H⁺═C₃₀H₅₆NO₁₀: 590.3898. Found: 590.3914. ¹³C NMR (125 MHz, CDCl₃): δ 220.7 (C-9), 175.0 (C-1), 106.7 (C-1′), 88.4 (C-5), 78.9 (C-3), 78.0 (C-6), 76.5 (C-13), 74.1 (C-12), 70.6 (C-2′), 70.2 (C-5′), 69.7 (C-11), 65.6 (C-3′), 49.5 (OMe), 45.5 (C-8), 44.5 (C-2), 40.2 (NMe₂), 38.7 (C-7), 37.5 (C-10), 35.8 (C-4), 28.0 (C-4′), 21.4 (C-14), 21.2 (Me at C-5′), 18.7 (Me at C-6), 17.7 (Me at C-8), 16.1 (Me at C-12), 15.2 (Me at C-2), 12.6 (Me at C-10), 10.4 (C-15), 8.2 (Me at C-4); MS, ESI pos. m/z (% rel. int.): 558.4 (8), 590.3 (100, [M+H⁺]), 612.4 (4, [M+Na⁺]).

### 3-O-(Allyloxycarbonyl)-N-demethyl-3-O-descladinosylclarithromycin 11,12-carbonate-2′,3′-urethane (10)

Phosgene (20% in toluene, 2.8 ml, 5.4 mmol) was added to a solution of the substrate 9 (545 mg, 0.92 mmol) in dichloromethane (12 ml) and pyridine (0.87 ml, 11 mmol). The reaction mixture was stirred at room temperature for 7 h before allyl alcohol (1.5 ml, 22 mmol) was added. The mixture was stirred for another 30 min, aqueous sodium hydroxide added, the mixture extracted with dichloromethane, the combined organic layers washed with water and brine, dried over magnesium sulfate, filtered and the filtrate evaporated. The residual yellow solid was purified by flash chromatography on silica gel (35 g) using hexane:ethyl acetate:triethylamine 49:49:2; yield 271 mg (41%) of the allyl carbonate 10 as a pale yellow solid. Recrystallisation from chloroform:hexane removed the discoloration and left a white solid; mp.: 290-295° C. (sublim.) (chloroform:hexane). (Calc. for C₃₅H₅₃NO₁₄: C, 59.06; H, 7.51. Found: C, 59.75; H, 8.08%). HRMS, ESI pos.: Calc. for M+Na⁺═C₃₅H₅₃NNaO₁₄: 734.3358. Found: 734.3349. See FIG. 4 for further Spectral Data.

### N-Demethyl-3-O-descladinosylclarithromycin 11,12-carbonate-2′,3′-urethane (7), from the allylic carbonate (10)

The allyl carbonate 10 (377 mg, 0.53 mmol) was dissolved in DMSO:THF (1:1, 12 ml) at 70° C. and 1,4-bis(diphenylphosphino)butane (dppb, 14 mg, 0.033 mmol) and bis(dibenzylideneacetone)palladium (Pd(dba)₂, 17 mg, 0.030 mmol, 6 mol %) were added. The reaction mixture was stirred at this temperature for 2 h, cooled to room temperature, aqueous sodium hydroxide added, the mixture extracted with dichloromethane, the combined organic layers washed with water and brine, dried over magnesium sulfate, filtered and the filtrate evaporated. The residual yellow solid (349 mg) was purified by flash chromatography on silica gel (30 g) using dichloromethane:isopropanol:triethylamine 97:1:2. The product was subsequently recrystallised from dichloromethane:toluene; yield 179 mg (54%) of the target compound 7 as a white solid. Analyses confirmed that the product was identical with the compound 7 prepared from the allyl carbonate 5 (vide supra).

### 10,11-Anhydro-N-demethylclarithromycin 2′,3′-urethane (11)

The carbonylated species 5 (500 mg, 0.58 mmol) was dissolved in DMSO (14 ml), and sodium azide (222 mg, 3.41 mmol) was added. The reaction mixture was stirred at 100° C. for 26 h. The resulting yellow solution was cooled to room temperature, aqueous sodium hydroxide added, and the cold reaction mixture extracted with ethyl acetate (PS: dichloromethane and NaN₃ form explosive geminal diazides). The combined organic layers were washed with water and brine, dried over magnesium sulfate and filtered. Evaporation of the filtrate left 441 mg of a pale yellow solid which was purified by flash chromatography on silica gel (22 g) using hexane:ethyl acetate:triethylamine 23:75:2; yield 381 mg (89%) of the conjugated enone 11 as a white solid; mp.: 157-160° C. (acetone:hexane). (Calc. for C₃₈H₆₃NO₁₃: C, 61.52; H, 8.56. Found: C, 60.81; H, 8.26%). HRMS, ESI pos.: Calc. for M+Na⁺═C₃₈H₆₃NNaO₁₃: 764.4191. Found: 764.4215. See FIG. 4 for further Spectral Data.

### 10,11-Anhydro-N-demethyl-3-O-descladinosylclarithromycin 2′,3′-urethane (12)

The substrate 11 (1.28 g, 1.73 mmol) was dissolved in acetic acid:water (1:1, 18 ml), and the reaction mixture was stirred at 70° C. for 1 h. The mixture was then cooled to room temperature and stirred for another 2 h. The precipitated solid was filtered off and dried in vacuo for 1 h; yield 661 mg (66%) of the 3-O-descladinosyl compound 12 as a white solid; mp.: 282-285° C. (dec.) (acetic acid:water). (Calc. for C₃₀H₄₉NO₁₀: C, 61.73; H, 8.46. Found: C, 61.63; H, 8.57%). HRMS, ESI pos.: Calc. for M+Na⁺═C₃₀H₄₉NNaO₁₀: 606.3248. Found: 606.3272. See FIG. 4 for further Spectral Data.

### 10,11-Anhydro-N-demethyl-3-O-descladinosyl-3-oxoclarithromycin 2′,3′-urethane (13)

The 3-hydroxy compound 12 (645 mg, 1.11 mmol) was dissolved in dichloromethane (20 ml), and Dess-Martin periodinane (DMP, 709 mg, 1.67 mmol) was added. The reaction mixture was stirred at room temperature for 30 min. Aqueous sodium hydroxide was added, and the reaction mixture was extracted with dichloromethane. The combined organic layers were washed with water and brine, dried over magnesium sulfate and filtered. Removal of the solvent afforded 742 mg of a white foam. Purification of the crude material by flash chromatography on silica gel (30 g) using dichloromethane:isopropanol:triethylamine 98:1:1 provided 603 mg (94%) of the β-keto ester 13 as a white solid; mp.: 231-234° C. (toluene:hexane). (Calc. for C₃₀H₄₇NO₁₀: C, 61.94; H, 8.14. Found: C, 61.74; H, 8.31%). HRMS, ESI pos.: Calc. for M+Na⁺═C₃₀H₄₇NNaO₁₀: 604.3092. Found: 604.3121. See FIG. 4 for further Spectral Data.

### 11-Amino-N-(4-bromophenyl )-N′-demethyl-11-deoxy-3-O-descladinosyl-3-oxoclarithromycin 11,12:2′,3′-diurethane (17)

The enone alcohol 13 (420 mg, 0.72 mmol) was dissolved in THF (12 ml), and sodium hydride (60% in mineral oil, 58 mg, 1.5 mmol) was added. The mixture was stirred at room temperature for 10 min before 4-bromophenyl isocyanate (433 mg, 2.19 mmol) and copper(I) chloride (80 mg, 0.81 mmol) were added. The mixture was stirred at 50° C. for 42 h, cooled to room temperature and quenched with sat. aq. ammonium chloride. Brine was added, the mixture extracted with THF, the combined organic layers washed with brine, dried over magnesium sulfate, filtered and the filtrate evaporated. The residual yellow solid (823 mg) was purified by flash chromatography on silica gel (42 g) using toluene:THF 84:16; yield 351 mg (62%) of the cyclic 11,12-urethane 17 as a white solid; mp.: 253-256° C. (diethyl ether). (Calc. for C₃₇H₅₁BrN₂O₁₁: C, 56.99; H, 6.59. Found: C, 56.03; H, 6.55%). HRMS, ESI pos.: Calc. for M+Na⁺═C₃₇H₅₁⁷⁹BrN₂NaO₁₁: 801.2568. Found: 801.2595. See FIG. 4 for further Spectral Data.

### 11-Amino-N-(3-fluoro-4-morpholinophenyl)-N′-demethyl-11-deoxy-3-O-descladinosyl-3-oxoclarithromycin 11,12:2′,3′-diurethane (19)

Sodium hydride (60% in mineral oil, 38 mg, 0.95 mmol) was added to a solution of the conjugated enone 13 (250 mg, 0.43 mmol) in THF (5 ml) and the mixture stirred at room temperature for 10 min before a solution of freshly prepared 3-fluoro-4-morpholinophenylisocyanate (1.29 mmol and copper(I) chloride (52 mg, 0.53 mmol) in THF (3 ml) was added. The reaction mixture was stirred in a sealed tube at 50° C. for 42 h. The mixture was cooled to room temperature, quenched with sat. aq. ammonium chloride and stirred for 1 h. Aqueous sodium hydroxide was added, and the mixture was extracted with ethyl acetate. The combined organic layers were washed with water and brine, dried over magnesium sulfate and filtered. The filtrate was evaporated to dryness leaving 463 mg of a pink solid. The crude product was purified by flash chromatography on silica gel (30 g) using dichloromethane:isopropanol:triethylamine 98:1:1; yield 193 mg (56%) of the title compound 19 as an off-white powder; mp.: 231-235° C. (acetone:diethyl ether). (Calc. for C₄₁H₅₈FN₃O₁₂: C, 61.26; H, 7.27. Found: C, 62.06; H, 7.24%). HRMS, ESI pos.: Calc. for M+H⁺═C₄₁H₅₉FN₃O₁₂: 804.4077. Found: 804.4103. See FIG. 4 for further Spectral Data.

### 11-Amino-N-(3-fluoro-4-morpholinophenyl)-11-deoxy-3-O-descladinosyl-3-oxoclarithromycin 11,12-carbamate (29)

Sodium hydride (60% in mineral oil, 38 mg, 0.95 mmol) is added to a solution of the conjugated enone 27 (250 mg, 0.43 mmol) in THF (5 ml) and the mixture stirred at room temperature for 10 min before a solution of freshly prepared 3-fluoro-4-morpholinophenylisocyanate (1.29 mmol and copper(I) chloride (52 mg, 0.53 mmol) in THF (3 ml) is added. The reaction mixture is stirred in a sealed tube at 50° C. for 42 h. The long reaction time is for allowing the reaction product to isomerise to a single stereoisomer. The reaction mixture at room temperature is quenched with sat. aq. ammonium chloride and stirred for 1 h. Aqueous sodium hydroxide is added, and the mixture extracted with ethyl acetate. The combined organic layers are washed with water and brine, dried over magnesium sulfate and filtered. Evaporation of the filtrate and purification of the residue by flash chromatography on silica gel using dichloromethane:isopropanol:triethylamine 98:1:1 furnish the title compound 29.

### 11-Amino-N-(3-fluoro-4-morpholinophenyl)-2′-O-benzoyl-11-deoxy-3-O-descladinosyl-3-oxoclarithromycin 11,12-carbamate (28)

Sodium bis(trimethylsilyl)amide (NaHMDS, ˜1 M in THF, 0.66 ml, 0.66 mmol) was added to a solution of the substrate (27, 200 mg, 0.30 mmol) in THF (4 ml). The mixture was stirred at room temperature under N₂ for 10 min. Copper(I) chloride (36 mg, 0.36 mmol) and a solution of 3-fluoro-4-morpholinophenyl isocyanate (0.89 mmol) in THF (2 ml) were added, and the reaction mixture was heated to 50° C. The mixture was stirred at this temperature for 42 h and cooled to room temperature. Aqueous sodium hydroxide (pH˜12, ca. 50 ml) was added, and the product was extracted into ethyl acetate (ca. 50 ml). The organic layer was washed with brine, dried with magnesium sulfate, filtered and concentrated. A black solid material (350 mg) was obtained. Flash chromatography on silica gel (20 g) using heptane/ethyl acetate/triethylamine 49:49:2 as the eluent provided 39 mg (17% yield) of the carbamate 28 as a yellow oil.

MS, ESI pos. m/z (% rel. int.): 674.7 (6, [substrate+H⁺]), 897.1 (100, [M+H⁺]), 912.6 (6), 1814.3 (4, [2M+Na⁺]).

### 11-Amino-N-(3-fluoro-4-morpholinophenyl)-11-deoxy-3-O-descladinosyl-3-oxoclarithromycin 11,12-carbamate (29)

The substrate (28, 35 mg, 0.039 mmol) was dissolved in methanol (1.5 ml) and stirred at 30° C. for 3 d and at 40° C. for 1 d. The mixture was diluted with ethyl acetate (ca. 20 ml), and dilute hydrochloric acid (pH˜1, ca. 20 ml) was added. The layers were separated, and aqueous sodium hydroxide was added to the aqueous phase until pH˜12. The product was extracted into ethyl acetate (ca. 30 ml), and the organic layer was washed with brine, dried with magnesium sulfate, filtered and concentrated. 18 mg (58% yield) of the C-2′ alcohol 29 were isolated as an off-white solid.

MS, ESI pos. m/z (% rel. int.): 570.9 (5), 793.3 (100, [M+H⁺]), 808.8 (10), 1361.5 (5), 1584.1 (8, [2M+H⁺]).

### REFERENCES

1. Hansen, L. H.; Mauvais, P.; Douthwaite, S. Mol. Microbiol. 1999, 31, 623-631.
2. Ma, Z.; Clark, R. F.; Brazzale, A.; Wang, S.; Rupp, M. J.; Li, L.; Griesgraber, G.; Zhang, S.; Yong, H.; Phan, L. T.; Nemoto, P. A.; Chu, D. T. W.; Plattner, J. J.; Zhang, X.; Zhong, P.; Cao, Z.; Nilius, A. M.; Shortridge, V. D.; Flamm, R.; Mitten, M.; Meulbroek, J.; Ewing, P.; Alder, J.; Or, Y. S. J. Med. Chem. 2001, 44, 4137-4156.
6. Fernandes, P. B.; Baker, W. R.; Freiberg, L. A.; Hardy, D. L.; McDonald, E. J. Antimicrob. Agents Chemother. 1989, 33, 78-81.
7. Flynn, E. H.; Murphy, H. W.; McMahon, R. E. J. Am. Chem. Soc. 1955, 77, 3104-3106.
8. Hunt, E.; Tyler, J. W. J. Chem. Soc., Perkin Trans. 2 1990, 2157-2162.
9. Martin, S. F.; Yamashita, M. J. Am. Chem. Soc. 1991, 113, 5478-5480.
10. Asaka, T.; Manaka, A.; Suglyama, H. Curr. Top. Med. Chem. 2003, 3, 961-989.
11. Corey, E. J.; Kim, C. U. J. Am. Chem. Soc. 1972, 94, 7586-7587.
12. Wu, Y.-J. Curr. Pharm. Des. 2000, 6, 181-223.
13. Agouridas, C.; Denis, A.; Auger, J.-M.; Benedetti, Y.; Bonnefoy, A.; Bretin, F.; Chantot, J.-F.; Dussarat, A.; Fromentin, C.; D'Ambrieres, S. G.; Lachaud, S.; Laurin, P.; Martret, O. L.; Loyau, V.; Tessot, N. J. Med. Chem. 1998, 41, 4080-4100.
14. Genet, J. P.; Blart, E.; Savignac, M.; Lemeune, S.; Lemair-Audoire, S.; Bernard, J. M. Synlett. 1993, 680-682.
15. Corey, E. J.; Weigel, L. O.; Floyd, D.; Bock, M. G. J. Am. Chem. Soc. 1978, 100, 2916-2918.
16. Baker, W. R.; Clark, J. D.; Stephens, R. L.; Kim, K. H. J. Org. Chem. 1988, 53, 2340-2345.
17. Dess, D. B.; Martin, J. C. J. Org. Chem. 1983, 48, 4155-4156.
18. Hartwig, J. F.; Angew. Chem. Int. Ed. 1998, 37, 2046-2067.
19. Hartwig, J. F.; Kawatsura, M.; Hauck, S. I.; Shaughnessy, K. H.; Alcazar-Roman, L. M.; J. Org. Chem. 1999, 64, 5575-5580.
20. Wolfe, J. P.; Buchwald, S. L. J. Org. Chem. 2000, 65, 1144-1157.
21. Kuwano, R.; Utsunomiya, M.; Hartwig, J. F. J. Org. Chem. 2002, 67, 6479-6486.
22. Brickner, S. J.; Hutchinson, D. K.; Barbachyn, M. R.; Manninen, P. R.; Ulanowics, D. A.; Garmon, S. A.; Grega, K. C.; Hendges, S. K.; Toops, D. S. J. Med. Chem. 1996, 39, 673-679.
23. Pearlman, B. A. WO 99/24393.
24. Freiberg, L. U.S. Pat. No. 3,725,385; Premchandran, R. US Pat. Appl. Nov. 22, 1996, case 6001.US.01.
25. Stenmark, H. G.; Brazzale, A.; Ma, Z. J. Org. Chem. 2000, 65, 3875-3876.
26. Hengeveld, J. E.; Gupta, A. K.; Kemp, A. H.; Thomas, A. V. Tetrahedron Lett. 1999, 40, 2497-2500.
27. Elliott, R. L.; Pireh, D.; Griesgraber, G.; Nilius, A. M.; Ewing, P. J.; Bul, M. H.; Raney, P. M.; Flamm, R. K.; Kim, K.; Henry, R. F.; Chu, D. T. W.; Plattner, J. J.; Or, Y. S. J. Med. Chem. 1998, 41, 1651-1659.
28. Or, Y. S.; Griesgraber, G.; Chu, D. T. W. WO 98/54197.

1. A macrolide of Formula I, and pharmaceutically acceptable polymorphs, salts, prodrugs and solvates thereof, 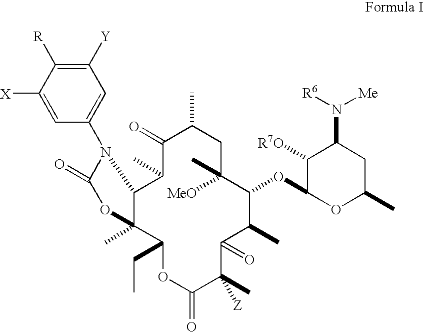
in which formula
X and Y independently represent hydrogen, hydroxy, cyano, carboxy, COOR¹³ (wherein R¹³ represents an optionally substituted aliphatic group), halogen, optionally substituted alkyl, optionally substituted alkoxy, or nitro, the benzene ring is optionally further substituted in the 2 and/or 6 position;
Z represents hydrogen or halogen;
R represents: (i) Morpholino, thiomorpholino, piperidino, or piperazino, said rings are optionally substituted on ring C atom(s) and/or ring N atom (such as with hydrogen, alkyl, alkoxy, —CO₂-alkyl, —CO-alkyl-OH, —CO-alkanediyl-OH, -alkanediyl-O-alkyl, -alkanediyl-halogen, -alkenyl-O-alkyl, -alkenyl-halogen (any said groups being optionally substituted));
(ii) optionally substituted alkanoyl;
(iii) —N(R⁸)R⁹, wherein R⁸ and R⁹ independently represent hydrogen or optionally substituted alkyl; or R⁸ and R⁹ together represent the group -alkanediyl-R¹¹-alkanediyl-, which group is optionally substituted, and R¹¹ represents CH₂, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl; or
(iv) —N(R⁸)R⁹, wherein R⁸ and R⁹ independently represent hydrogen or optionally substituted alkyl; or R⁸ and R⁹ together represent the group -alkenyl-R¹¹-alkenyl-, which group is optionally substituted, and R¹¹ represents CH₂, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl;
R represents, together with the vicinal X:
(i) —CO-alkanediyl-, which is optionally substituted;
(ii) —NR⁵-alkanediyl-, which is optionally substituted and wherein R⁵ represents optionally substituted alkanoyl;
(iii) —CO-alkenyl-, which is optionally substituted
(iv) —NR⁵-alkenyl-, which is optionally substituted and wherein R⁵ represents optionally substituted alkanoyl;
R⁶ represents hydrogen or optionally substituted alkyl; and R⁷ represents hydrogen, a hydroxy-protecting group or optionally substituted alkanoyl; or R⁶ and R⁷ together represent —CO—.

2. A macrolide according to claim 1, wherein
X and Y independently represent hydrogen, hydroxy, cyano, carboxy, COOR¹³ (wherein R¹³ represents C₁₋₆ alkyl), halogen, C₁₋₆ alkyl, C₁₋₆ alkoxy, or nitro;
Z represents hydrogen or halogen;
R represents: (i) Morpholino, thiomorpholino, piperidino, or piperazino (optionally substituted with halogen, C₁₋₆ alkyl, C₁₋₆ alkoxy, —CO₂—C₁₋₆-alkyl, —CO—C₁₋₆ alkanediyl-OH, —C₁₋₆ alkanediyl-O—C₁₋₆ alkyl, —C₁₋₆ alkanediyl-halogen); or
(ii) C₁₋₆ alkanoyl, optionally substituted with hydroxy;
(iii) —N(R⁸)R⁹, wherein R⁸ and R⁹ independently represent hydrogen or C₁₋₆ alkyl; or R⁸ and R⁹ together represent the group —C₁₋₆ alkanediyl-R¹¹—C₁₋₆ alkanediyl-, and R¹¹ represents CH₂, S, O, SO, SO₂, SNR¹², S(O)NR¹² or NR¹², wherein R¹² represents hydrogen or C₁₋₆ alkyl (optionally substituted with halogen, hydroxy, C₁₋₆ alkoxy, amino, C₁₋₆ alkylamino, or di-C₁₋₆ alkylamino) or p-toluenesulfonyl; or
R represents, together with the vicinal X:
(i) —CO—C₁₋₆-alkanediyl-; or
(ii) —NR⁵—C₁₋₆-alkanediyl-, wherein R⁵ represents C₁₋₆ alkanoyl;
R⁶ represents hydrogen or C₁₋₆ alkyl; and R⁷ represents hydrogen, a hydroxy-protecting group or C₁₋₆ alkanoyl; or R⁶ and R⁷ together represent —CO—.

3. A macrolide according to claim 1, wherein
X and Y independently represent hydrogen, halogen, or C₁₋₆ alkoxy;
Z represents hydrogen or halogen;
R represents: (i) Morpholino, thiomorpholino, or piperazino substituted at N-4 by R⁴, said rings are optionally substituted by R² and R³ which independently represent hydrogen, halogen, or C₁₋₆ alkoxy; R⁴ represents hydrogen, —CO₂—(C₁₋₆ alkyl), —COCH₂OH, —CH₂CH₂O—(C₁₋₆ alkyl), —CH₂CH₂F; or
(ii) C₁₋₆ alkanoyl optionally substituted by at least one hydroxyl group; or
R represents, together with the vicinal X:
(i) —CO(CH₂)ₙ— wherein n represents the integer 2 or 3; or
(ii) —NR⁵CH₂CH₂— where R⁵ represents C₁₋₆ alkanoyl optionally substituted by at least one hydroxyl group;
R⁶ represents hydrogen or C₁₋₆ alkyl; and R⁷ represents hydrogen or C₁₋₆ alkanoyl; or R⁶ and R⁷ together represent —CO—.

4. A macrolide according to claim 1, wherein
X and Y independently represent hydrogen, halogen, such as fluor, or C₁₋₆ alkoxy, such as methoxy;
Z represents hydrogen or halogen, such as fluor or chlor;
R represents: (i) Morpholino, thiomorpholino, or piperazino substituted at N-4 by R⁴, said rings are optionally substituted by R² and R³ which independently represent hydrogen, halogen, such as fluor or chlor, or C₁₋₆ alkoxy, such as methoxy; R⁴ represents hydrogen, —CO₂—(C₁₋₆ alkyl), —COCH₂OH, —CH₂CH₂O—(C₁₋₆ alkyl), —CH₂CH₂F; or
(ii) C₁₋₆ alkanoyl optionally substituted by at least one hydroxyl group, such as —COCH₂OH, i.e. indoline derivatives; or
R represents, together with the vicinal X:
(i) —CO(CH₂)ₙ— wherein n represents the integer 2 or 3, i.e. indanone and tetralone derivatives; or
(ii) —NR⁵CH₂CH₂— where R⁵ represents C₁₋₆ alkanoyl optionally substituted by at least one hydroxyl group;
R⁶ represents hydrogen or C₁₋₆ alkyl, such as methyl; and R⁷ represents hydrogen or C₁₋₆ alkanoyl; or R⁶ and R⁷ together represent —CO—, i.e. oxazolidinone derivatives.

5-13. (canceled)

14. A macrolide according to claim 1 selected from the following:
(a) 11-amino-N-(3-fluoro-4-morpholinophenyl)-11-deoxy-3-O-descladinosyl-3-oxoclarithromycin 11,12-oxazolidinone
(b) 11-Amino-N-(3-fluoro-4-morpholinophenyl)-N′-demethyl-11-deoxy-3-O-descladinosyl-3-oxoclarithromycin 11,12:2′,3′-diurethane (19).
(c) 11-Amino-N-(3-fluoro-4-morpholinophenyl)-11-deoxy-3-O-descladinosyl-3-oxoclarithromycin 11,12-carbamate, and
(d) 11-Amino-N-(3-fluoro-4-morpholinophenyl)-2′-O-benzoyl-11-deoxy-3-O-descladinosyl-3-oxoclarithromycin 11,12-carbamate

15-17. (canceled)

18. A pharmaceutical composition comprising a compound according to claim 1, together with a pharmaceutically acceptable carrier or excipient.

19. A method for treatment of an animal (such as a mammal, including a human), said method comprising administering a pharmaceutical composition according to claim 18 to the animal.

20. A process for the preparation of an 11-deoxy-11-aminomacrolide of claim 1 wherein the 11-amino and the 12-oxy groups are joined by a common carbonyl group to form an annulated oxazolidin-2-one which is N11-arylated by an optionally substituted phenyl ring, and/or pharmaceutically acceptable salts, prodrugs and/or solvates thereof, said process comprises reacting a 11-deoxy-11-deoxy-macrolide with a phenylisocyanate, which is optionally substituted on the phenyl ring.

21. The process of claim 18, wherein CuCl and/or NaH are used as reagents, and/or the phenyl ring in the phenylisocyanate carries an electron withdrawing substituent, such as a halogen atom.

22. A process for the preparation of a macrolide of claim 1, said process comprises:
reacting the corresponding 11-deoxy-macrolide with a 3-Y, 4-R, 5-X phenylisocyanate (wherein Y, R and X are defined as above); or
reacting the corresponding 11-deoxy-macrolide with CDI and 3-Y, 4-R, 5-X aniline (wherein Y, R and X are defined as above); or
reacting the corresponding 11-deoxy-11,12-urethane macrolide with a 3-Y, 4-R, 5-X phenyl halide (wherein Y, R and X are defined as above);
and/or
deprotecting a protected derivative of a macrolide of formula I (e.g. a macrolide of formula I wherein R7 is a protecting group); and/or
converting a macrolide of formula I to an other macrolide of formula I; and/or
reacting a macrolide of formula I with a pharmaceutically acceptable acid or base; and/or
crystallizing a macrolide of formula I in a suitable solvent.

23. Use of a compound according to claim 1 for the manufacture of a pharmaceutical composition, such as an antibacterial composition.

24-25. (canceled)

26. A macrolide according to claim 1, wherein
X and Y independently represent hydrogen, hydroxy, cyano, carboxy, COOR¹³ (wherein R¹³ represents an optionally substituted aliphatic group), halogen, optionally substituted alkyl, optionally substituted alkoxy, or nitro, the benzene ring is optionally further substituted in the 2 and/or 6 position;
Z represents hydrogen or halogen;
R represents: (i) Morpholino, thiomorpholino, piperidino, or piperazino, said rings are optionally substituted on ring C atom(s) and/or ring N atom (such as with hydrogen, alkyl, alkoxy, —CO₂-alkyl, —CO-alkyl-OH, -alkenyl-O-alkyl, -alkenyl-halogen (any said groups being optionally substituted)); or
(ii) optionally substituted alkanoyl;
(iii) —N(R⁸)R⁹, wherein R⁸ and R⁹ independently represent hydrogen or optionally substituted alkyl; or R⁸ and R⁹ together represent the group -alkenyl-R¹¹-alkenyl-, which group is optionally substituted, and R¹¹ represents CH2, S, O, SO, SO₂, SNR¹², S(O)NR¹² or NR¹², wherein R¹² represents hydrogen or optionally substituted alkyl (such as with halogen, hydroxy, alkoxy, amino, alkylamino, or dialkylamino) or p-toluenesulfonyl; or
R represents, together with the vicinal X:
(i) —CO-alkenyl-, which is optionally substituted; or
(ii) —NR⁵-alkenyl-, which is optionally substituted and wherein R⁵ represents optionally substituted alkanoyl;
R⁶ represents hydrogen or optionally substituted alkyl; and R⁷ represents hydrogen, a hydroxy-protecting group or optionally substituted alkanoyl; or R⁶ and R⁷ together represent —CO—.
